(19) United States
(12) Patent Application Publication
(10) Pub. No.: US 2016/0074207 A1
(43) Pub. Date: Mar. 17, 2016

(54) ORAL APPLIANCE

(71) Applicants: Hyun-Jin Choi, Incheon (KR); Young-Hyon Park, Incheon (KR); Jun-Young Park, Incheon (KR); Park Jun-Won, Incheon (KR)

(72) Inventors: Hyun-Jin Choi, Incheon (KR); Young-Hyon Park, Incheon (KR); Jun-Young Park, Incheon (KR); Park Jun-Won, Incheon (KR)

(21) Appl. No.: 14/949,016

(22) Filed: Nov. 23, 2015

Related U.S. Application Data
(63) Continuation-in-part of application No. 14/762,600, filed on Jul. 22, 2015.

(30) Foreign Application Priority Data
Jan. 23, 2013 (KR) 1020130007414
Dec. 10, 2013 (KR) 1020130153414
Oct. 22, 2015 (KR) 1020150147532

(51) Int. Cl. A61F 5/56

(52) U.S. Cl. CPC A61F 5/566

(57) ABSTRACT

There is provided an oral appliance including: a body having a shape corresponding to a teeth arrangement; an upper teeth recess formed in an upper portion of the body to allow teeth included in the teeth arrangement of an upper jaw to be inserted therein; and a lower teeth recess formed in a lower portion of the body to allow teeth included in the teeth arrangement of a lower jaw to be inserted therein and allowing the lower jaw to move forward by a predetermined distance.

### CROSS-REFERENCE TO RELATED APPLICATIONS

The present case claims the benefit of the filing date of Korean Application No. KR 10-2015-0147532, filed on Oct. 22, 2015. This case is also a continuation-in-part, and claims the benefit of the filing date of U.S. patent application Ser. No. 14/762,600, filed Jul. 22, 2015, which is a U.S. National Stage application claiming priority to PCT Application No. PCT/KR2014/000681 filed Jan. 23, 2014. PCT Application No. PCT/KR2014/000681 claims the benefit of the filing dates of Korean Application No. KR 10-2013-0007414, filed Jan. 23, 2013, and Korean Application No. KR 10-2013-0153414, filed Dec. 10, 2013. The entire contents of each of the aforementioned patent applications are incorporated herein by reference.

### TECHNICAL FIELD

The present disclosure relates to an oral appliance installed in teeth arrangements of upper and lower jaws in an oral cavity and allowing a lower jaw to move forward by a predetermined distance to maintain an open state of airway, and more particularly to an oral appliance in which at least one of an upper teeth recess and a lower teeth recess is spaced apart from teeth inserted therein at a predetermined interval in vertical and horizontal directions.

### BACKGROUND ART

During sleep, a person's tongue often relaxes backward within an oral cavity. This relaxation can restrict the person's airway, causing the airway to become narrowed.

In this state, inhaled air may not entirely enter the narrowed airway and instead remain in the oral cavity. Also, residual air vibrates soft tissues around the airway to generate noise, which is commonly known as snoring.

Snoring may be frequently accompanied by obstructive sleep apnea.

In order to prevent snoring and sleep apnea, a CPAP (Constant Positive Airway Press device), which forcibly supplies air to the airway during sleep, may be used. However, while such a CPAP may be effective for snoring and sleep apnea, many people find it to be inconvenient to use and costly, and the CPAP sometimes leads to certain side effects.

Also, a surgical intervention to remove drooped soft tissues around such as palatopharynx, pharynx and soft palate may be considered, but this is not favorably accepted because recurrence is common and side effects can be expected.

Other solutions include the use of an oral appliance that is installed in teeth arrangements of both the upper and lower jaws within an oral cavity to prevent snoring and sleep apnea. These oral appliances force the lower jaw to be maintained forward, causing the tongue, which is attached to the lower jaw, to be forwardly pulled out, thereby relieving pressure applied to the airway by the tongue and widening the airway to allow air to flow smoothly.

These oral appliances come in two varieties: an integrated type oral appliance and an separated type oral appliance.

The integrated type oral appliance is formed by integrating an upper jaw teeth insertion part into which teeth included in a teeth arrangement of an upper jaw are inserted and a lower jaw teeth insertion part into which teeth included in a teeth arrangement of a lower jaw are inserted.

In contrast, the separated type oral appliance is formed by separating the upper jaw teeth insertion part and the lower jaw teeth insertion part.

The integrated type oral appliance has the upper jaw teeth insertion part and lower jaw teeth insertion part integrated, as mentioned above.

Thus, when the integrated type oral appliance is used, the lower jaw cannot move during sleeping after the oral appliance is inserted, causing the lower jaw to move forward by a predetermined distance. This can cause the TMJ (Temporo-Mandibular Joint), neighboring muscles or a ligaments to be stiffened or for pain to be present.

In addition, in the case of the separated type oral appliance, the upper jaw teeth insertion part and the lower jaw teeth insertion part are separated to allow the lower jaw to move during sleep, as mentioned above. Also, the upper jaw teeth insertion part and the lower jaw teeth insertion part are connected by a connection member to allow the lower jaw to move.

However, even with the separated type oral appliance, a movement of the lower jaw is restrained by the connection member that is affixed to the lower jaw, and the aforementioned problem remains unsolved.

### DISCLOSURE OF INVENTION

### Technical Problem

The present disclosure has been devised through recognition of any one of the issues or problems evident in the related art.

An aspect of the present disclosure provides an oral appliance that allows a lower jaw to move even while the user is wearing the oral appliance.

An aspect of the present disclosure also provides an oral appliance allowing stiffness of related muscles and ligaments, caused by a forward movement of a lower jaw, to be relaxed by enabling a lower jaw to move.

An aspect of the present disclosure also provides an oral appliance capable of preventing pain, malocclusion, or a TMJ disorder from occurring even while a user is wearing the oral appliance.

### Solution to Problem

An oral appliance, in relation to an exemplary embodiment for performing at least one of the foregoing tasks, may have the following features.

According to an aspect of the present disclosure, there is provided an oral appliance including: a body having a shape corresponding to a teeth arrangement; an upper teeth recess formed in an upper portion of the body to allow teeth included in the teeth arrangement of an upper jaw to be inserted therein; and a lower teeth recess formed in a lower portion of the body to allow teeth included in the teeth arrangement of a lower jaw to be inserted therein and allowing the lower jaw to move forward by a predetermined distance.

The oral appliance may further include a guide part extending from the body in at least one or more of upward and downward directions by a predetermined distance to thereby guide teeth of the lower jaw or upper jaw to be inserted into the upper teeth recess or the lower teeth recess.

The guide part may extend from an inner side of the body.

The guide part may extend to cover at least a portion of a gum part of molars among the teeth.

The teeth of the upper jaw may be fixed in the upper teeth recess through friction, and the lower teeth recess may be spaced apart from the teeth of the lower jaw at predetermined intervals in vertical and horizontal directions.

A gap between the lower teeth recess and the teeth of the lower jaw in the vertical direction may range from 0.5 mm to 2 mm.

A gap between the lower teeth recess and the teeth of the lower jaw in the horizontal direction in one side thereof may range from 0.3 mm to 0.5 mm.

The teeth of the lower jaw may be fixed in the lower teeth recess through friction, and the upper teeth recess may be spaced apart from the teeth of the upper jaw at predetermined intervals in vertical and horizontal directions.

A gap between the upper teeth recess and the teeth of the upper jaw in the vertical direction may range from 0.5 mm to 2 mm.

A gap between the upper teeth recess and the teeth of the upper jaw in the horizontal direction in one side thereof may range from 0.3 mm to 0.5 mm.

The upper teeth recess may be spaced apart from the teeth of the upper jaw at a predetermined interval in vertical and horizontal directions, and the lower teeth recess may be spaced apart from the teeth of the lower jaw at a predetermined interval in vertical and horizontal directions.

A gap between the upper teeth recess and the teeth of the upper jaw in the vertical direction and a gap between the lower teeth recess and the teeth of the lower jaw in the vertical direction may range from 0.5 mm to 2 mm, and a gap between the upper teeth recess and the teeth of the upper jaw in the horizontal direction in one side thereof and a gap between the lower teeth recess and the teeth of the lower jaw in the horizontal direction in one side thereof may range from 0.3 mm to 0.5 mm.

### Advantageous Effects of Invention

As described above, according to an exemplary embodiment of the present disclosure, since a space is formed between an upper teeth recess or a lower teeth recess, to which teeth are inserted, and the teeth in vertical and horizontal directions in the oral appliance, when a user wears the oral appliance, a lower jaw may move.

Also, according to an exemplary embodiment of the present disclosure, stiffness of related muscles and ligaments caused by a forward movement of the lower jaw may be relaxed as a movement of the lower jaw is allowed.

In addition, according to an exemplary embodiment of the present disclosure, even if the oral appliance is worn, pain, malocclusion, or a TMJ disorder does not occur, unlike the related art oral appliance.

Moreover, according to an exemplary embodiment of the present disclosure, since air flow through an airway is facilitated, rhinitis or rhinosinusitis may be mitigated.

### BRIEF DESCRIPTION OF DRAWINGS

FIG. 1 includes perspective views of an oral appliance according to a first exemplary embodiment of the present disclosure, in which (a) is a top perspective view and (b) is a bottom perspective view.

FIG. 2 is a cross-sectional view taken along line A-A′ in (a) of FIG. 1.

FIG. 3 is a view illustrating the oral appliance worn in an oral cavity according to the first exemplary embodiment of the present disclosure, in which (a) is a front view and (b) is a side view.

FIG. 4 is a cross-sectional view and a partially enlarged cross-sectional view of (b) of FIG. 3.

FIG. 5 is a cross-sectional view and a partially enlarged cross-sectional view illustrating a usage state of the oral appliance according to the first exemplary embodiment of the present disclosure.

FIG. 6 is a cross-sectional view and a partially enlarged cross-sectional view illustrating a wearing state of the oral appliance according to a second exemplary embodiment of the present disclosure.

FIG. 7 is a cross-sectional view and a partially enlarged cross-sectional view illustrating a wearing state of the oral appliance according to a third exemplary embodiment of the present disclosure.

FIG. 8 is a perspective cross-sectional view illustrating an oral appliance according to a fourth exemplary embodiment of the present disclosure.

FIG. 9 illustrates a fifth embodiment of the invention.

FIG. 10 is another view of the appliance in accordance with the fifth embodiment.

FIG. 11 illustrates the fifth embodiment from a top-down angle (viewed from above the upper jaw recess).

FIG. 12 illustrates the fifth embodiment viewed from the front.

FIG. 13 illustrates the fifth embodiment viewed from the side.

FIG. 14 illustrates a sixth embodiment of the invention.

### MODE FOR THE INVENTION

To help understand the foregoing features of the present disclosure, an oral appliance related to an exemplary embodiment of the present disclosure will be described in detail.

Exemplary embodiments of the present disclosure will now be described in detail with reference to the accompanying drawings.

The disclosure may, however, be exemplified in many different forms and should not be construed as being limited to the specific embodiments set forth herein. Rather, these embodiments are provided so that this disclosure will be thorough and complete, and will fully convey the scope of the disclosure to those skilled in the art.

In the drawings, the shapes and dimensions of elements may be exaggerated for clarity, and the same reference numerals will be used throughout to designate the same or like elements.

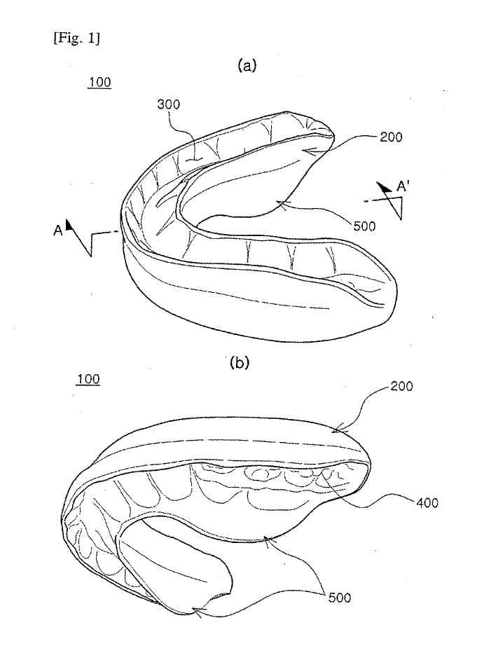

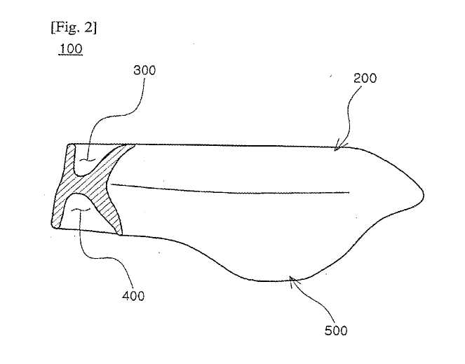

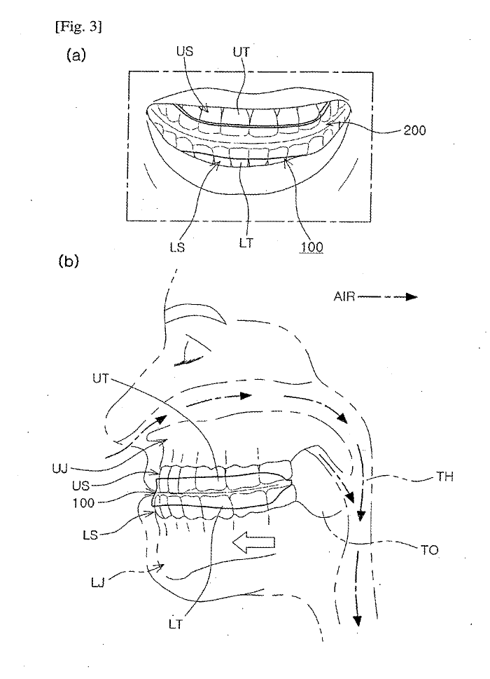

FIG. 1 includes perspective views of an oral appliance according to a first exemplary embodiment of the present disclosure, in which (a) is a top perspective view and (b) is a bottom perspective view, FIG. 2 is a cross-sectional view taken along line A-A′ in (a) of FIG. 1, and FIG. 3 is a view illustrating the oral appliance worn in an oral cavity according to the first exemplary embodiment of the present disclosure, in which (a) is a front view and (b) is a side view.

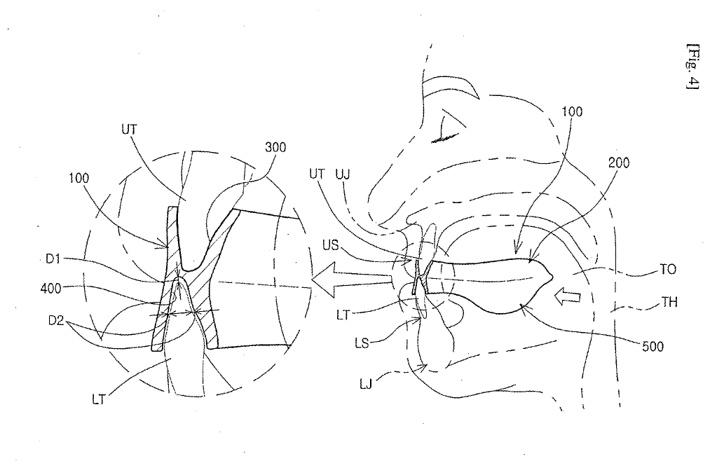

FIG. 4 is a cross-sectional view and a partially enlarged cross-sectional view of (b) of FIG. 3, and FIG. 5 is a cross-sectional view and a partially enlarged cross-sectional view illustrating a usage state of the oral appliance according to the first exemplary embodiment of the present disclosure.

An oral appliance according to the exemplary embodiment of the present disclosure may include a body 200, an upper teeth recess 300, a lower teeth recess 400, and a guide part 500.

As illustrated in FIGS. 1 and 2, the body 200 may have a shape corresponding to teeth arrangements US and LS. Namely, the body 200 may have a horseshoe shape. Thus, the teeth arrangement US of an upper jaw UJ and the teeth arrangement LS of a lower jaw LJ may correspond to the body 200.

As illustrated in FIGS. 3 and 4, teeth UT included in the teeth arrangement US of the upper jaw UJ may be inserted into the upper teeth recess 300 formed in an upper portion of the body 200 as described hereinafter, and teeth LT included in the teeth arrangement LS of the lower jaw LJ may be inserted into the lower teeth recess 400 formed in a lower portion of the body 200 as described hereinafter.

A material of the body 200 is not particularly limited and may be any material as long as it does not pose a threat when the body 200 formed of the material is applied to an oral cavity. Examples of this material include but are not limited to one or more of: a bioresin former by monomer and polymer; bioresin block for a computer-aided design/computer-aided manufacturing (CAD/CAM) device; a U.S. Food and Drug Administration approved material used by three-dimensional (3D) printers; and/or resin. The molar-covering portions of the body are typically slightly shorter than a user's actual molars and extend only part of the distance from the top of the molars to the gumline.

In order to manufacture the oral appliance 100 according to an exemplary embodiment of the present disclosure including the body 200, any known method may be used, including but not limited to: forming a model of the teeth UT and LT of a user and subsequently shaping the oral appliance 100 appropriately based on the model, taking an image of the teeth UT and LT of the user and manufacturing the oral appliance 100 by using a 3D printer, or the like.

The upper teeth recess 300 may be formed in an upper portion of the body 200. As illustrated in FIGS. 3 through 5, the teeth UT included in the teeth arrangement US of the upper jaw UJ may be inserted into the upper teeth recess 300. To this end, the upper teeth recess 300 may have a shape corresponding to the teeth arrangement US of the upper jaw UJ on the whole, namely, a horseshoe shape as mentioned above; also, the upper teeth recess 300 may have a shape corresponding to each tooth UT included in the teeth arrangement US of the upper jaw UJ.

A method for forming the upper teeth recess 300 in the upper portion of the body 200 is not particularly limited, and any method, such as forming a model of the teeth UT of a user and subsequently shaping the upper teeth recess 300 to correspond thereto or forming the upper teeth recess 300 by using a 3D printer, or the like, may be used.

In any case, in an embodiment of the invention, the teeth UT of the upper jaw UJ may be fixed to the upper teeth recess 300 through friction. Namely, as mentioned above, the upper teeth recess 300 may be formed to have a shape corresponding to the teeth UT of the upper jaw UJ such that there is no gap between the upper teeth recess 300 and the teeth UT of the upper jaw UJ. For example, the upper teeth recess 300 can be formed in such a way that the upper teeth recess 300 uses certain UTs in the mouth of the wearer as anchor points for fixing the upper teeth recess 300 to the UT. In an embodiment, the upper teeth recess 300 can be anchored to each of the backmost molars of the UT. In the front of the UT, upper teeth recess 300 can be anchored by friction to any or all of the canines, incisors and/or bicuspids of the wearer.

Thus, the teeth UT of the upper jaw UJ may be tightly inserted into the upper teeth recess 300 and fixed to the upper teeth recess 300 through friction. Unless an external force stronger than frictional force between the upper teeth recess 300 and the teeth UT of the upper jaw UJ is applied, the teeth UT of the upper jaw UJ will not be separated from the upper teeth recess 300.

The lower teeth recess 400 may be formed in a lower portion of the body 200. As illustrated in FIGS. 3 through 5, the teeth LT included in the teeth arrangement LS of the lower jaw LJ may be inserted into the lower teeth recess 400. To this end, the lower teeth recess 400 may have a shape corresponding to the teeth arrangement LS of the lower jaw LJ on the whole, namely, a horseshoe shape as mentioned above; also, the lower teeth recess 400 may have a shape corresponding to each tooth LT included in the teeth arrangement LS of the lower jaw LJ. However, in contrast to the tight insertion of the UT into the upper teeth recess, the LT are only loosely inserted into the lower teeth recess 400. Thus, the alignment of the UT and LT can be maintained by oral appliance while the jaw of the wearer is relaxed (e.g., while the wearer is asleep. However, the LT can be easily removed from the lower teeth recess 400, such as in cases where the wearer begins to feel jaw discomfort.

To this extent, the method for forming the lower teeth recess 400 in the lower portion of the body 200 is not particularly limited, and any method may be used, including, but not limited to, forming a model of the teeth LT of a user and subsequently shaping the lower teeth recess 400 to generally correspond thereto, taking an image of the teeth LT of a user and forming the lower teeth recess 400 based on the image by using a 3D printer, or the like.

When the teeth LT of the lower jaw LJ is inserted into the lower teeth recess 400, the lower jaw LJ may move forward by a predetermined distance. Namely, as illustrated in FIGS. 3 and 4, the lower jaw LJ may move forward by a predetermined distance A such that front teeth of the lower jaw LJ are positioned in front of the front teeth of the upper jaw UJ by a predetermined distance, ranging from, for example, 1 mm to 12 mm, depending on the size of the wearer's mouth and teeth. Accordingly, a tongue TO, being attached to the lower jaw LJ, may also move forward along with the lower jaw LJ, as illustrated.

Thus, as illustrated in FIGS. 3 and 4, the tongue TO may not block the airway TH and the airway TH may also be widened. Also, as illustrated in (b) of FIG. 3, air may flow smoothly through the airway TH. Thus, snoring, occurring due to the tongue TO blocking the airway TH and the airway being narrowed during sleep, may be prevented. Also, sleep apnea occurring together with snoring may be prevented. Also, rhinitis or rhinosinusitis may be mitigated.

To this end, as mentioned above, the lower teeth recess 400 may be formed in the body 400 such that when the teeth LT of the lower jaw LJ is inserted, the teeth LT of the lower jaw LJ move forward relative to a natural position, such that the front teeth of the lower jaw LJ are positioned in front of the front teeth of the upper jaw UJ by a predetermined distance A.

Meanwhile, as illustrated in FIG. 4, when the teeth LT of the lower jaw LJ are inserted into the lower teeth recess 400, the teeth LT of the lower jaw LJ may be spaced apart from the lower teeth recess 400 at predetermined gaps D1 and D2 therebetween in vertical and horizontal directions.

With the presence of the gaps D1 and D2 between the lower teeth recess 400 and the teeth LT of the lower jaw LJ in the vertical and horizontal directions, as illustrated in FIG. 5, the teeth LT of the lower jaw LJ may be released from the lower teeth recess 400 and moved downwards as needed; accordingly, the lower jaw LJ may also be moved downwards.

Thus, since the lower jaw LJ may naturally move downwards, stiffness of the neighboring muscles and ligaments occurred by, for example, the forward movement of the lower jaw LJ and maintenance of this state during sleep, may be resolved and relaxed.

The foregoing downward movement of the lower jaw LJ may be stopped when the guide part 500 comes into contact with a lingual mucous membrane of the lower jaw LJ. When this state is maintained, the neighboring muscles and ligaments may stiffen again.

In order to resolve this, the lower jaw LJ may naturally move upwards, namely, may return to a position before the movement, in which the teeth LT of the lower jaw LJ are inserted into the lower teeth recess 400 and the lower jaw LJ moves forward.

The downward movement of the lower jaw LJ from the oral appliance 100 and the upward movement thereof to the oral appliance 100 may be repeated while the user, wearing the oral appliance 100 according to the exemplary embodiment of the present disclosure, is sleeping.

Thus, even if the user wears the oral appliance 100 according to the exemplary embodiment of the present disclosure for an extended period of time in his or her sleep every night, the muscles and ligaments around the oral cavity may not be stiffened. Also, pain, malocclusion, or a TMJ disorder caused by rigidity of the muscles and ligaments around the oral cavity may not occur. Also, since air flow through the airway is improved, rhinitis or rhinosinusitis may be mitigated.

The gap D1 between the teeth LT of the lower jaw LJ and the lower teeth recess 400 in the vertical direction may range from 0.5 mm to 2 mm.

According to released research, a natural gap between the teeth UT of the upper jaw UJ and the teeth LJ of the lower jaw LJ, for example, the gap during sleep, ranges approximately from 2 mm to 4 mm.

Meanwhile, a distance between the upper teeth recess 300 and the lower teeth recess 400 is approximately 1.5 mm. Thus, if the gap D1 between the lower teeth recess 400 and the teeth LT of the lower jaw LJ in the vertical direction is smaller than 0.5 mm, a distance between the teeth UT of the upper jaw UJ and the teeth LT of the lower jaw may be smaller than 2 mm to 4 mm, the natural gap.

In this case, the brain may misunderstand the body 200 interposed between the teeth UT of the upper jaw UJ and the teeth LT of the lower jaw LJ to be food, causing masticatory movements to be made by the related muscles and ligaments. Force exerted by the masticatory movements may be transmitted to the teeth UT and LT of the upper and lower jaws UJ and LJ through the oral appliance 100 according to the exemplary embodiment of the present disclosure.

The force transmitted to the teeth UT and LT may break the teeth UT and LT or periodontal ligaments and destroy an alveolar bone. Also, the teeth UT and LT may be lost or periodontitis may develop. Namely, the teeth UT and LT may be damaged.

In addition, the force exerted by the masticatory movements may spread to the muscles, ligaments, and TMJ around the oral cavity having the oral appliance 100 according to the exemplary embodiment of the present disclosure installed therein, damaging the muscles and ligaments. As a result, malocclusion or a TMJ disorder, as along with pain, may occur.

Meanwhile, if the gap D1 between the lower teeth recess 400 and the teeth LT of the lower jaw LJ in the vertical direction exceeds 2 mm, a distance between the teeth UT of the upper jaw UJ and the teeth LT of the lower jaw may be greater than 4 mm, the natural gap.

Thus, the related muscles and ligaments may be burdened and stiffened. Also, pain, malocclusion, and a TMJ disorder may occur.

Therefore, the gap D1 between the lower teeth recess 400 and the teeth LT of the lower jaw LJ in the vertical direction ranging from 0.5 mm to 2 mm is appropriate.

Meanwhile, the gap D2 between the lower teeth recess 400 and the teeth LT of the lower jaw LJ in the horizontal direction may range from 0.3 mm to 0.5 mm.

Also, the lower teeth recess 400 may be spaced apart from the teeth LT of the lower jaw LJ on both sides thereof. As discussed above, since the gap D2 between the lower teeth recess 400 and the teeth LT of the lower jaw LJ in the horizontal direction on one side of the lower teeth recess 400 ranges from 0.3 mm to 0.5 mm, a total gap in both sides may range from 0.6 mm to 1.0 mm.

If the gap D2 between the lower teeth recess 400 and the teeth LT of the lower jaw LJ in the horizontal direction on one side of the lower teeth recess 400 is smaller than 0.3 mm, the teeth LT of the lower jaw LJ may occasionally be fixed to the lower teeth recess 400 through friction. Also, the teeth LT of the lower jaw LJ may be hindered from being released and moving downwards from the lower teeth recess 400, when needed.

Thus, the related muscles and ligaments stiffened due to the forward movement of the lower jaw LJ may not be relaxed, causing pain, discomfort, malocclusion, or a TMJ disorder.

Meanwhile, when the gap D2 between the lower teeth recess 400 and the teeth LT of the lower jaw LJ in the horizontal direction on one side of the lower teeth recess 400 exceeds 0.5 mm, the teeth LT of the lower jaw LJ may be freely moved within the lower teeth recess 400, but the teeth LT of the lower jaw LJ may be completely released and separated from the lower teeth recess 400.

Thus, the lower jaw LJ may not be maintained in a state in which it is placed forward by a predetermined distance, losing the ability to prevent snoring or sleep apnea.

Therefore, the gap D2 between the lower teeth recess 400 and the teeth LT of the lower jaw LJ in the horizontal direction on one side of the lower teeth recess 400 ranging from 0.3 mm to 0.5 mm is appropriate.

The range of the gaps D1 and D2 between the lower teeth recess 400 and the teeth LT of the lower jaw LJ in the vertical or horizontal direction may be modified to be individually applied by adding or subtracting a value from a reference value of a portion or entirety of the lower teeth recess 400 according to a tilt angle of the occlusal plane, a degree of protrusion of teeth, an inclination state of teeth in forward/backward and inward/outward directions, or a degree of teeth deviating from the overall teeth arrangement, of each of the users.

Also, as illustrated in FIG. 4, the lower teeth recess 400 may be spaced apart from the teeth LT of the lower jaw LJ in the horizontal direction forwardly and backwardly. In addition, the lower teeth recess 400 may also be spaced apart from the teeth LT of the lower jaw LJ in the horizontal direction left and right.

As illustrated in FIGS. 1 and 2, the guide part 500 may extend downwards from the body 200, wherein downwards is relative to a position of the appliance when worn in a mouth of the wearer. For example, the guide part 500 may extend downwards by 3 mm to 5 mm. The guide part 500 may have a curved configuration extending from the back molar area. However, the length of the guide part 500 extending from the body 200 may not be particularly limited and any length may be applied according to a state of an oral cavity of individuals.

As described above, as the teeth LT of the lower jaw LJ move downwards from the lower teeth recess 400 to relax the related muscles and ligaments stiffened as the lower jaw LJ moves forward and maintained in the state of being ahead by a predetermined distance, the lower jaw LJ may also be moved downwards by a predetermined distance naturally.

Such movement of the lower jaw LJ may be stopped when the guide part 500 comes into contact with the lingual mucous membrane of the lower jaw LJ, and the lower jaw LJ may be maintained in that state.

When the lower jaw LJ is moved or maintained in a moved state to relax the stiffened muscles and ligaments, other normal muscles or ligaments may be stiffened. In order to relax the other stiffened muscles and ligaments, the lower jaw LJ may be naturally moved upwards as illustrated in FIG. 5, and the teeth LT of the lower jaw LJ may be inserted into the lower teeth recess 400.

At this time, the guide part 500 may guide the teeth LT of the lower jaw LJ to be easily and properly inserted into the lower teeth recess 400.

To this end, the guide part 500 may extend from an inner side of the body 200 as illustrated in FIGS. 1 and 2. Also, the guide part 500 may extend to cover a portion of a gum part of the molars among the teeth LT of the lower jaw LJ.

However, the shape of the guide part 500 is not particularly limited and the guide part 500 may have any shape as long as it has the aforementioned functions.

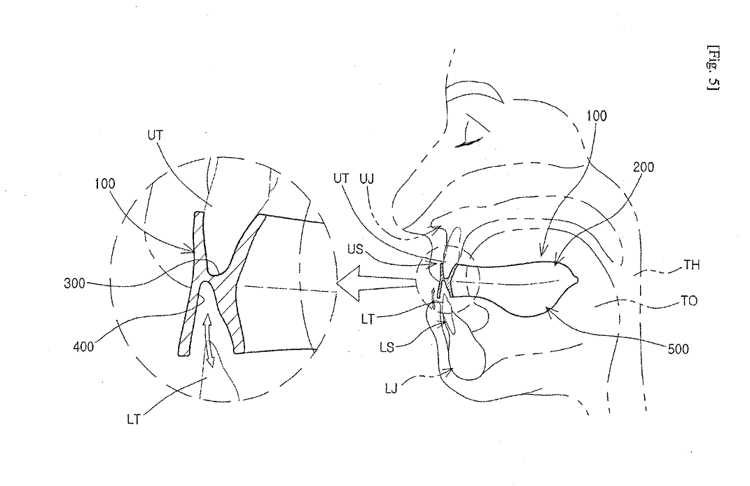

FIG. 6 is a cross-sectional view and a partially enlarged cross-sectional view illustrating a wearing state of the oral appliance according to a second exemplary embodiment of the present disclosure. The oral appliance 1100 according to the second exemplary embodiment of the present disclosure is different from that of the first exemplary embodiment described above with reference to FIGS. 1 through 5 in that, when the oral appliance is worn, the teeth LT of the lower jaw LJ are fixed in the lower teeth recess 1400 through friction, the upper teeth recess 1300 is spaced apart from the teeth UT of the upper jaw UJ at predetermined intervals D3 and D4 in vertical and horizontal directions, and guide parts 1500 and 1550 extends upwards from the body 1200. Thus, different parts and components will mainly be described, and the other components may simply refer back to the above descriptions with reference to FIGS. 1 through 5.

As illustrated in FIG. 6, in the oral appliance 100 according to the second exemplary embodiment of the present disclosure, the teeth UT of the lower jaw LJ may be fixed in the lower teeth recess 400. Namely, the lower teeth recess 400 may be formed to correspond to the teeth LT of the lower jaw LJ such that there is no gap between the lower teeth recess 400 and the teeth LT of the lower jaw LJ. For example, the lower teeth recess 400 can be formed in such a way that the lower teeth recess 400 uses certain LTs in the mouth of the wearer as anchor points for fixing the lower teeth recess 400 to the LT. In an embodiment, the lower teeth recess 400 can be anchored to each of the backmost molars of the LT. In the front of the LT, lower teeth recess 400 can be anchored by friction to any or all of the canines, incisors and/or bicuspids of the wearer.

Thus, the teeth LT of the lower jaw LJ may be tightly inserted into the lower teeth recess 400 and fixed through friction. Also, the teeth LT of the lower jaw LJ will not be separated from the lower teeth recess 400 unless an external force greater than frictional force between the lower teeth recess 400 and the teeth LT of the lower jaw LJ is applied.

The upper teeth recess 300 may be spaced apart from or loosely spaced with respect to the teeth LT of the upper jaw UJ at predetermined intervals D1 and D2 in the vertical and horizontal directions. To this extent, in contrast to the tight insertion of the LT into the lower teeth recess, the UT are only loosely inserted into the upper teeth recess 300. Thus, the alignment of the LT and UT can be maintained by oral appliance while the jaw of the wearer is relaxed (e.g., while the wearer is asleep). However, the UT can be easily removed from the upper teeth recess 300, such as in cases where the wearer begins to feel jaw discomfort.

Through this configuration, as the teeth LT of the upper jaw UJ are released from the upper teeth recess 300 and relatively moved in order to relax related muscles and ligaments stiffened due to a forward movement of the lower jaw LJ, the lower jaw LJ may naturally move downwards together with the oral appliance 100 according to the present exemplary embodiment.

While the lower jaw LJ is moving downwards together with the oral appliance 100, if the guide part 500 comes into contact with the molars among the teeth LT of the upper jaw UJ released from the upper teeth recess 300, the lower jaw LJ may stop moving and be maintained in the same state.

In this state, in order to relax other stiffened muscles and ligaments, the lower jaw LJ may naturally move upwards together with the oral appliance 100 according to the present exemplary embodiment to the position, namely, to the position prior to being released, in which the teeth LT of the upper jaw UJ are inserted into the upper teeth recess 300 and the lower jaw LJ moves forward. That is, the lower jaw LJ may be returned.

At this time, the guide part 500 may guide the teeth LT of the upper jaw UJ to be easily and properly inserted into the upper teeth recess 300.

The reciprocating movement of the lower jaw LJ may be repeated while the user wearing the oral appliance 100 according to the exemplary embodiment of the present disclosure is sleeping.

Thus, as described above, the related muscles and ligaments stiffened due to the forward movement of the lower jaw and the movement of the lower jaw may be relaxed, and thus, even though the user wears the oral appliance, pain, malocclusion, or a TMJ disorder may not occur. Also, since air flow through the airway is facilitated, rhinitis or rhinosinusitis may be mitigated.

Meanwhile, the gap D1 between the upper teeth recess 300 and the teeth LT of the upper jaw UJ in the vertical direction may range from 0.5 mm to 2 mm. Also, the gap D2 between the upper teeth recess 300 and the teeth LT of the upper jaw UJ in the horizontal direction in one side thereof may range from 0.3 mm to 0.5 mm.

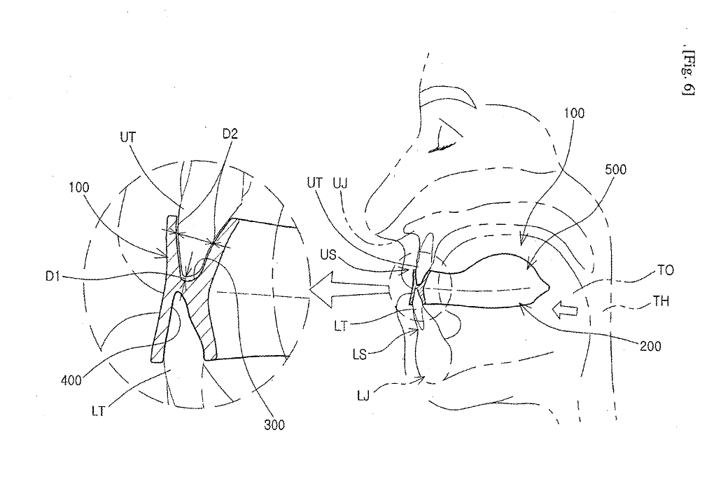

FIG. 7 is a cross-sectional view and a partially enlarged cross-sectional view illustrating a wearing state of an oral appliance according to a third exemplary embodiment of the present disclosure.

The oral appliance 100 according to the third exemplary embodiment of the present disclosure is different from that of the first exemplary embodiment, described above with reference to FIGS. 1 through 5, in that the upper teeth recess 300 is spaced apart from the teeth UT of the upper jaw UJ at predetermined intervals D1 and D2 in vertical and horizontal directions, the lower teeth recess 400 is spaced apart from the teeth UT of the lower jaw LJ at predetermined intervals D1 and D2 in vertical and horizontal directions, and the guide part 500 extends downwards and upwards from the body 200.

Thus, different parts and components will mainly be described, and the other components may simply refer back to the above descriptions with reference to FIGS. 1 through 5.

As illustrated in FIG. 7, in the oral appliance 100 according to the third exemplary embodiment of the present disclosure, the upper teeth recess 300 may be spaced apart from the teeth UT of the upper jaw UJ at predetermined intervals D1 and D2 in the vertical and horizontal directions, and lower teeth recess 400 may be spaced apart from the teeth LT of the lower jaw LJ at predetermined intervals D1 and D2 in the vertical and horizontal directions.

Also, the guide part 500 may extend both upwards and downwards by a predetermined distance.

The oral appliance 100 according to the third exemplary embodiment of the present disclosure may be used in a case in which a state of the teeth arrangements US and LS of the upper and lower jaws UJ and LJ or a state of the teeth UT and LT included therein is so poor, such that the teeth may be lost if fixed to the upper teeth recess 300 or the lower teeth recess 400 through friction, in a case in which a length of the crowns of the upper and lower jaws is too short to obtain sufficient frictional force.

In this configuration, when any one of the teeth UT of the upper jaw UJ and the teeth LT of the lower jaw LJ is more tightly attached to the upper teeth recess 300 or the lower teeth recess 400 according to posture during sleep, a side less tightly attached may be moved within the upper teeth recess 300 or the lower teeth recess 400. Thus, the lower jaw LJ may be naturally moved to relax the stiffened muscles and ligaments.

For example, if the teeth UT of the upper jaw UJ are more tightly attached to the upper teeth recess 300, the lower jaw LJ may perform a reciprocating movement in a manner identical to that of the oral appliance 100 according to the first exemplary embodiment of the present disclosure as described above, and if the teeth UT of the lower jaw LJ are more tightly attached to the lower teeth recess 400, the lower jaw LJ may perform a reciprocating movement in a manner identical to that of the oral appliance 100 according to the second exemplary embodiment of the present disclosure as described above.

The reciprocating movement of the lower jaw LJ may be repeated while the user wearing the oral appliance 100 according to the exemplary embodiment of the present disclosure is sleeping.

Thus, as described above, the related muscles and ligaments stiffened due to the forward movement of the lower jaw and the other movement of the lower jaw may be relaxed, and thus, even though the user wears the oral appliance, pain, malocclusion, or a TMJ disorder may not occur. Also, since air flow through the airway is facilitated, rhinitis or rhinosinusitis may be mitigated.

In this case, the gap D1 between the upper teeth recess 300 and the teeth LT of the upper jaw UJ in the vertical direction and the gap D2 between the lower teeth recess 400 and the teeth LT of the lower jaw LJ in the vertical direction may range from 0.5 mm to 2 mm.

Also, the gap D2 between the upper teeth recess 300 and the teeth LT of the upper jaw UJ in the horizontal direction in one side thereof, and the gap D2 between the lower teeth recess 400 and the teeth LT of the lower jaw LJ in the horizontal direction in one side thereof may range from 0.3 mm to 0.5 mm.

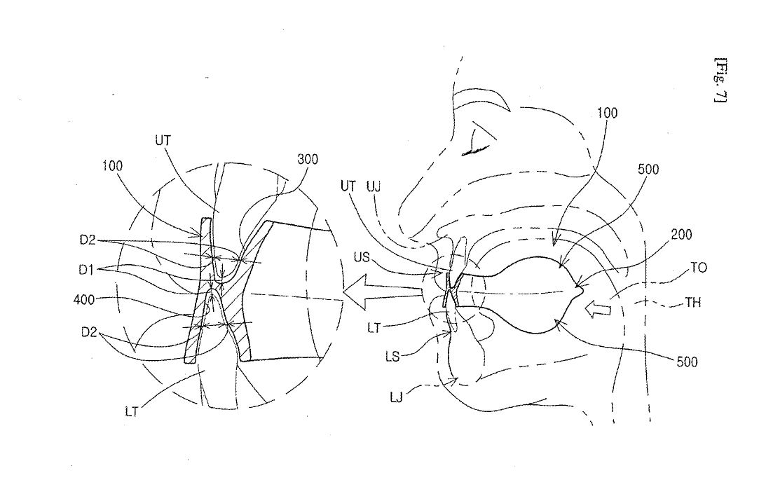

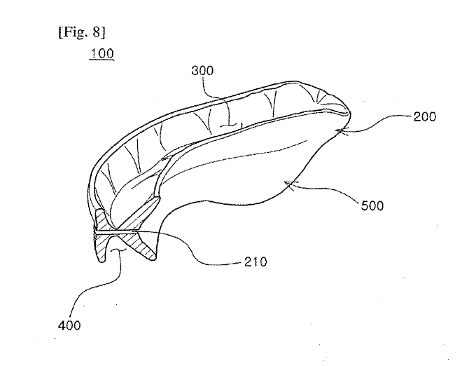

FIG. 8 is a perspective cross-sectional view illustrating an oral appliance according to a fourth exemplary embodiment of the present disclosure.

The oral appliance 100 according to the fourth exemplary embodiment of the present disclosure is different from that of the first exemplary embodiment described above with reference to FIGS. 1 through 5, in that a vent hole 210 is formed in the body 200 of the oral appliance 100.

Thus, different parts and components will mainly be described, and the other components may simply refer back to the above descriptions with reference to FIGS. 1 through 5.

As illustrated in FIG. 8, in the oral appliance 100 according to the fourth exemplary embodiment of the present disclosure, the vent hole 210 may be formed in the body 200. The vent hole 210 may be formed in a central portion of the body 200 between the upper teeth recess 300 and the lower teeth recess 400.

Thus, the user may breathe through his or her mouth through the vent hole 210 of the body 200 while wearing the oral appliance 100 according to the fourth exemplary embodiment of the present disclosure.

As described above, in the case of using the oral appliance according to the exemplary embodiment of the present disclosure, even if a user wears the oral appliance, a lower jaw may naturally move, and related muscles and ligaments stiffened due to a forward movement of the lower jaw may be relaxed as the lower jaw is allowed to move. In addition, although a user wears the oral appliance, pain, malocclusion, or a TMJ disorder may not occur, and since air flow through an airway is facilitated, rhinitis or rhinosinusitis may be mitigated.

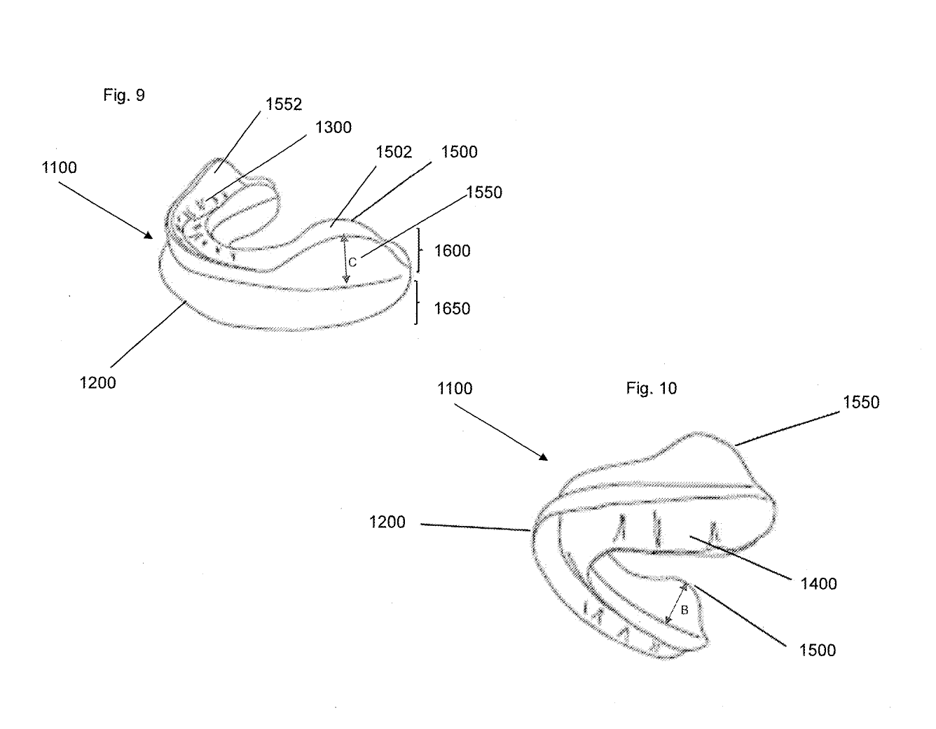

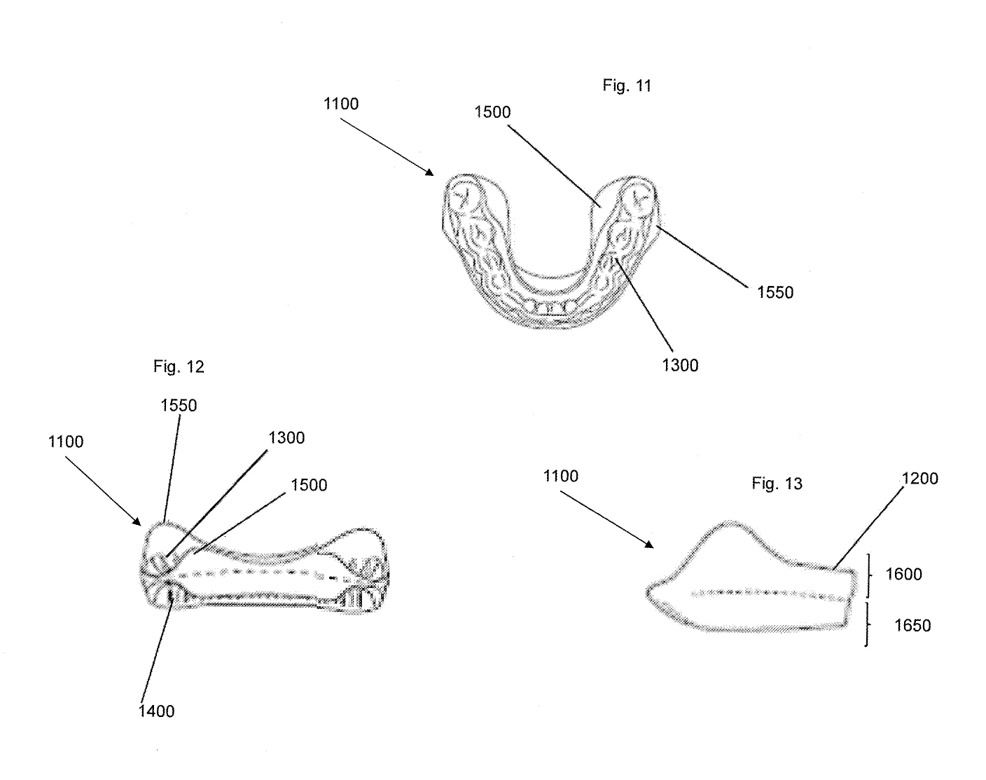

FIGS. 9 through 13 illustrate a fifth embodiment of the invention. Like the second embodiment, the lower teeth recess of the oral appliance is fixed to the lower teeth LT using friction. FIG. 9 is a view of the appliance in accordance with the fifth embodiment. FIG. 10 is another view of the appliance in accordance with the fifth embodiment. FIG. 11 illustrates the fifth embodiment from a top-down angle (viewed from above the upper jaw recess). FIG. 12 illustrates a front view of the fifth embodiment. FIG. 13 illustrates a side view of the fifth embodiment.

The fifth view will now be discussed in reference to FIGS. 9-13. As shown, an oral appliance according to the fifth exemplary embodiment of the present disclosure may include a body 1200, an upper teeth recess 1300, a lower teeth recess 1400, an inner guide part 1500, and an outer guide part 1550. Upper teeth recess 1300 includes: an inner guide part 1500 that is adjacent to the back of the UT when the oral appliance 1100 is being worn by the user, and an outer guide part 1550 that is adjacent to the front of the UT when the appliance 1100 is being work by the user. The guide parts 1500 and 1550 may extend upwards from the body 1200 of oral appliance 1100. In an embodiment, guide part 1500 is angled relatively backward from the vertical with respect to the UT. In any case, guide parts 1500 and 1550 provide guides for ensuring the UT are aligned properly with respect to LT when oral appliance 1100 is being worn and the wearer's jaw is in the closed position.

The body 1200 may have a shape corresponding to teeth arrangements US and LS. Namely, the body 1200 may have a horseshoe shape. Thus, the teeth arrangement US of an upper jaw UJ and the teeth arrangement LS of a lower jaw LJ may correspond to the body 1200.

Teeth UT included in the teeth arrangement US of the upper jaw UJ may be inserted into the upper teeth recess 1300 formed in an upper portion of the body 1200 as described hereinafter, and teeth LT included in the teeth arrangement LS of the lower jaw LJ may be inserted into the lower teeth recess 1400 formed in a lower portion of the body 1200 as described hereinafter.

A material of the body 1200 is not particularly limited and may be any material as long as it does not pose a threat when the body 1200 formed of the material is applied to an oral cavity. Examples of this material include but are not limited to one or more of: a bioresin former by monomer and polymer; bioresin block for a CAD/CAM device; a U.S. Food and Drug Administration approved material used by 3D printers; and/or resin. The molar-covering portions of the body are typically slightly shorter than a user's actual molars and extend only part of the distance from the top of the molars to the gumline.

In order to manufacture the oral appliance 1100 according to an exemplary embodiment of the present disclosure including the body 1200, any known method may be used, including but not limited to: forming a model of the teeth UT and LT of a user and subsequently shaping the oral appliance 1100 appropriately based on the model, or taking an image of the teeth UT and LT of the user, and manufacturing the oral appliance 1100 by using a 3D printer, or the like.

The lower teeth recess 1400 may be formed in a lower portion of the body 1200. Teeth LT included in the teeth arrangement LS of the lower jaw LJ may be inserted into the lower teeth recess 1400. To this end, the lower teeth recess 1400 may have a shape corresponding to the teeth arrangement LS of the lower jaw LJ on the whole, namely, a horseshoe shape as mentioned above; also, the upper teeth recess 1400 may have a shape corresponding to each tooth LT included in the teeth arrangement LS of the lower jaw LJ.

A method for forming the lower teeth recess 1400 in the lower portion of the body 1200 is not particularly limited, and any method, such as forming a model of the lower teeth LT of a user and subsequently shaping the lower teeth recess 1400 to correspond thereto or forming the lower teeth recess 1400 by using a 3D printer, or the like.

In any case, in an embodiment of the invention, the teeth LT of the lower jaw LJ may be fixed to the lower teeth recess 1400 through friction. Namely, as mentioned above, the lower teeth recess 1400 may be formed to have a shape corresponding to the teeth LT of the lower jaw LJ such that there is no gap between the lower teeth recess 1400 and the teeth LT of the lower jaw LJ. For example, the lower teeth recess 1400 can be formed in such a way that the lower teeth recess 1400 uses certain LTs in the mouth of the wearer as anchor points for fixing the lower teeth recess 1400 to the LT. In an embodiment, the lower teeth recess 1400 can be anchored to each of the backmost molars of the LT. In the front of the LT, lower teeth recess 1400 can be anchored by friction to any or all of the canines, incisors and/or bicuspids of the wearer.

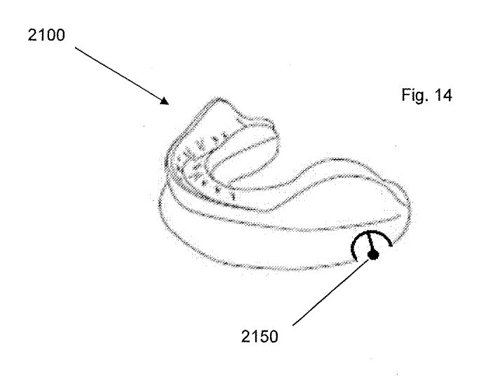

Thus, the teeth LT of the lower jaw LJ may be tightly inserted into the lower teeth recess 1400 and fixed to the lower teeth recess 1400 through friction. Unless external force is stronger than frictional force between the lower teeth recess 1400 and the teeth LT of the lower jaw LJ is applied, the teeth LT of the lower jaw LJ will not be separated from the lower teeth recess 1400. As shown in FIG. 14, in some embodiments, rather than being held just by friction of the teeth with the lower teeth recess 1400, the oral appliance 2100 is additionally or instead, held to the teeth LT by a metal wire clasp 2150 present on each side of the appliance 2100. Although such clasp 2150 is only shown on one side of the appliance 2100, in embodiments, another such wire clasp may be present on the other side of the appliance 2100, as well.

The upper teeth recess 1300 may be formed in an upper portion of the body 1200. The teeth UT included in the teeth arrangement US of the upper jaw UJ may be inserted into the upper teeth recess 1300. To this end, the upper teeth recess 1300 may have a shape corresponding to the teeth arrangement US of the upper jaw UJ on the whole, namely, a horseshoe shape as mentioned above; also, the upper teeth recess 1300 may have a shape corresponding to each tooth UT included in the teeth arrangement US of the upper jaw UJ. However, in contrast to the tight insertion of the LT into the lower teeth recess, the UT are only loosely inserted into the upper teeth recess 1300. Thus, the alignment of the UT and LT can be maintained by oral appliance while the jaw of the wearer is relaxed (e.g., while the wearer is asleep). However, the UT can be easily removed from the upper teeth recess 1300, such as in cases where the wearer begins to feel jaw discomfort.

A method for forming the upper teeth recess 1300 in the upper portion of the body 1200 is not particularly limited, and any method, such as forming a model of the teeth UT of a user and subsequently shaping the upper teeth recess 1300 to correspond thereto, or forming the upper teeth recess 1300 by using a 3D printer, or the like. To this extent, the method for forming the upper teeth recess 1300 in the upper portion of the body 1200 is not particularly limited, and any method may be used, including, but not limited to, forming a model of the teeth UT of a user and subsequently shaping the upper teeth recess 1300 to generally correspond thereto, or taking an image of the teeth UT of a user and forming the lower teeth recess 1300 based on the image by using a 3D printer, or the like. Like the embodiment shown in FIG. 6, the upper teeth recess 1300 may be spaced apart from the teeth LT of the upper jaw UJ at predetermined intervals D1 D3 and D2 D4 in the vertical and horizontal directions, respectively.

The guide parts 1500 and 1550 may extend upward from the body 1200. The guide parts 1500 and 1550 may extend upwards from the body 1200, wherein upwards is relative to a position of the appliance when worn in a mouth of the wearer. The guide parts 1500 and 1550 may have a curved configuration extending about a distance of B and C respectively, each measuring approximately 5 mm to 20 mm. The guide starts from the distal surface of the upper cranial and is curved through the first to the second molar. However, if there is a wisdom tooth present, then it extends to the wisdom tooth. However, the length of the guide part 1500 extending from the body 1200 may not be particularly limited and any length may be applied according to a state of an oral cavity of individuals.

To this end, the guide parts 1500 and 1550 may extend from an inner side of the body 1200 and an outer side of the body 1200, respectively. Also, the guide parts 1500 and 1550 may extend to cover a portion of a gum part of the molars among the teeth UT of the lower jaw UJ. However, the shape of the guide parts 1500 and 1550 is not particularly limited and the guide parts 1500 and 1550 may have any shape as long they are configured to produce the functionality as described herein.

When the oral appliance is attached, the lower jaw is forced forward, which causes tension to the ligaments and muscles near the temporomandibular joints. As time passes, the tension increases and the lower jaw tends to return back to the original position. Therefore, the lower jaw opens and moves down. Because the appliance is fixed to the lower jaw (especially to the lower molar portion), the appliance also moves with the lower jaw as well and gets out of the upper jaw. Due to the discomfort of open lower jaw after a while, the patient tends to let the lower jaw move to more comfortable position.

The guides 1500 and 1550 guide the upper teeth UT to the desired position when the lower jaw LJ closes back to the upper jaw UJ. When the lower jaw closes, the upper teeth UT contact and slide along the inner surface 1552 of the outer guide 1550 and the outer surface 1502 of inner guide 1500, which makes the upper jaw UJ disposed at the forwarded position.

The guides 1500 and 1550 further prevent the lower jaw LJ to open to the side direction too much. If it opens too much, that is, over the height of the appliance, the patient may undesirably bite the appliance. This may harm the teeth, ligaments, muscles and/or the appliance. To prevent these accidents, as shown on FIG. 10, the guides 1500 and 1550 are elongated to the direction to the (upper) alveolar bone so that the patient can't bite the appliance with open mouth. The space for moving to side directions inside the upper body 1600, helps the stress of the ligaments and muscles decrease.

The inner guide 1500 is configured not to interfere the movement of a wearer's tongue. The inner guide 1500 is shorter in height than the outer guide 1550. If it were longer than shown the figure, the tongue is interfered when moving forwards or upwards. (When the patient breathes through the nose instead of the (undesirable) mouth, the tongue needs to move upwards.)

The inner guide 1500 guides auxiliarily the upper teeth to the desired position when the lower jaw closes back. When the lower jaw LJ closes, the upper teeth UT are supposed to contact the inner surface of the outer guide 1550 and the outer surface of the inner guide 1500 and slide along to the upper teeth recess. In reality, the lower jaw LJ abruptly bounces back when the upper UT contact the inner surface. The inner guide 1500 helps the lower jaw LJ to stop moving backwards, which in the end makes the LJ be disposed at the forwarded position. (Think of the bobsleigh hitting the sidewall of the lane and returning to the center of the lane.)

Through this configuration, as the teeth UT of the upper jaw UJ are released from the upper teeth recess 1300 and relatively moved in order to relax related muscles and ligaments stiffened due to a forward movement of the lower jaw LJ, the lower jaw LJ may naturally move downwards together with the oral appliance 1100 according to the present exemplary embodiment. Lower teeth LT are held by the oral appliance 1100 in the lower teeth recess 1400 of lower body 1650.

While the lower jaw LJ is moving downwards together with the oral appliance 1100, if the guide parts 1500 and 1550 comes into contact with the molars among the teeth LT of the upper jaw UJ released from the upper teeth recess 1300, the lower jaw LJ may stop moving and be maintained in the same state.

In this state, in order to relax other stiffened muscles and ligaments, the lower jaw LJ may naturally move upwards together with the oral appliance 1100 according to the present exemplary embodiment to the position, namely, to the position prior to being released, in which the teeth UT of the upper jaw UJ are inserted into the upper teeth recess 1300 and the lower jaw LJ moves forward. That is, the lower jaw LJ may be returned.

At this time, the guide parts 1500 and 1550 may guide the teeth UT of the upper jaw UJ to be easily and properly inserted into the upper teeth recess 1300. Thus, like illustrated in FIG. 3, the tongue TO may not block the airway TH and the airway TH may also be widened. Also, as illustrated in (b) of FIG. 8, air may flow smoothly through the airway TH. Thus, snoring, occurring due to the tongue TO blocking the airway TH and the airway being narrowed during sleep, may be prevented. Also, sleep apnea occurring together with snoring may be prevented. Also, rhinitis or rhinosinusitis may be mitigated.

The reciprocating movement of the lower jaw LJ may be repeated while the user wearing the oral appliance 100 according to the exemplary embodiment of the present disclosure is sleeping.

Thus, as described above, the related muscles and ligaments stiffened due to the forward movement of the lower jaw and the movement of the lower jaw may be relaxed, and thus, even though the user wears the oral appliance, pain, malocclusion, or a TMJ disorder may not occur. Also, since air flow through the airway is facilitated, rhinitis or rhinosinusitis may be mitigated.

Meanwhile, like shown in FIG. 6, the gap D1 D3 between the upper teeth recess 1300 and the teeth LT UT of the upper jaw UJ in the vertical direction may range from 0.5 mm to 2 mm. Also, the gap D2 D4 between the upper teeth recess 300 and the teeth LT of the upper jaw UJ in the horizontal direction in one side thereof may range from 0.3 mm to 0.5 mm.

The range of the gaps D1 and D2 between the lower teeth recess 400 and the teeth LT of the lower jaw LJ in the vertical or horizontal direction may be modified to be individually applied by adding or subtracting a value from a reference value of a portion or entirety of the lower teeth recess 400 according to a tilt angle of the occlusal plane, a degree of protrusion of teeth, an inclination state of teeth in forward/backward and inward/outward directions, or a degree of teeth deviating from the overall teeth arrangement, of each of the users.

The upper teeth recess 1300 may be spaced apart from the teeth UT of the upper jaw UJ in the horizontal direction forwardly and backwardly. In addition, the upper teeth recess 1300 may also be spaced apart from the teeth UT of the upper jaw UJ in the horizontal direction left and right.

As described above, in the case of using the oral appliance according to the exemplary embodiment of the present disclosure, even if a user wears the oral appliance, a lower jaw may naturally move, and related muscles and ligaments stiffened due to a forward movement of the lower jaw may be relaxed as the lower jaw is allowed to move. In addition, although a user wears the oral appliance, pain, malocclusion, or a TMJ disorder may not occur, and since air flow through an airway is facilitated, rhinitis or rhinosinusitis may be mitigated.

The oral appliance as described above is not limited in its application to the configurations of the foregoing exemplary embodiments, but the entirety or a portion of the exemplary embodiments can be selectively combined to configure various modifications.

1. An oral appliance comprising:
a body having a shape corresponding to a teeth arrangement;
an upper teeth recess formed in an upper portion of the body to allow teeth included in the teeth arrangement of an upper jaw to be inserted therein; and
a lower teeth recess formed in a lower portion of the body to allow teeth included in the teeth arrangement of a lower jaw to be inserted therein and allowing the lower jaw to move forward by a predetermined distance;
wherein, when the oral appliance is worn by a user, the teeth of the lower jaw is fixed in the lower teeth recess through friction, and
wherein, when the oral appliance is worn by a user, the upper teeth recess is spaced apart from the teeth of the upper jaw at predetermined intervals in vertical and horizontal directions.

2. The oral appliance of claim 1, further comprising a guide part extending from the body in at least one or more of upward and downward directions by a predetermined distance to thereby guide teeth of the lower jaw or upper jaw to be inserted into the upper teeth recess or the lower teeth recess.

3. The oral appliance of claim 2, wherein the guide part extends from an inner side of the body.

4. The oral appliance of claim 2, wherein the guide part extends to cover at least a portion of a gum part of molars among the teeth.

5. The oral appliance of claim 1, wherein a gap between the upper teeth recess and the teeth of the upper jaw in the vertical direction ranges from 0.5 mm to 2 mm.

6. The oral appliance of claim 1, wherein a gap between the upper teeth recess and the teeth of the upper jaw in the horizontal direction in one side thereof ranges from 0.3 mm to 0.5 mm.

7. The oral appliance of claim 1, wherein a gap between the upper teeth recess and the teeth of the upper jaw in the horizontal direction in one side thereof is over 1 mm.
